(19) United States
(12) Patent Application Publication
(10) Pub. No.: US 2019/0076284 A1
(43) Pub. Date: Mar. 14, 2019

(54) SHOULDER BRACE

(71) Applicant: COLUMBIA HEALTHCARE CO., LTD., Seoul (KR)

(72) Inventors: Jeong Ran KWARK, Gwangmyeong-si (KR); Mi Seon Lee, Hanam-si (KR)

(21) Appl. No.: 15/796,964

(22) Filed: Oct. 30, 2017

(30) Foreign Application Priority Data
Sep. 11, 2017 (KR) 10-2017-0116132

(51) Int. Cl. A61F 5/01

(52) U.S. Cl. CPC A61F 5/013; A61F 2005/0155; A61F 2005/0158

(57) ABSTRACT

Provided is a shoulder brace having a forearm part, an upper-arm part, a torso part, an elbow-joint part, and a shoulder-joint part. The upper-arm part includes a rotary annulus to be coupled to an outside of the shoulder-joint part, the rotary annulus having line-shaped protrusions and grooves that are alternately formed along an inner circumference thereof. The shoulder-joint part includes a joint shaft, a push button provided on a first end thereof and coupled with the joint shaft, a locking ring coupled to a middle portion of the joint shaft and having line-shaped protrusions and grooves that are alternately formed along an outer circumference thereof, and a stopper formed on a second end thereof and providing space to allow the joint shaft to be movable when the push button is pushed, thus allowing a lock state to be released.

### CROSS-REFERENCE(S) TO RELATED APPLICATION

This application claims priority of Korean Patent Application No. 10-2017-0116132, filed on 11 Sep. 2017, in the Korean Intellectual Property Office, which is hereby incorporated by reference in its entirety.

### BACKGROUND OF THE INVENTION

### Field of the Invention

The present invention relates to a shoulder brace. More particularly, the invention relates to a shoulder brace, which guarantees easy wearing, is adjustable in length depending on the body condition of a wearer, and is capable of limiting or adjusting the motion ranges of the shoulder joint and the elbow joint depending on the condition of a wearer's shoulder.

### Description of the Related Art

Generally, the shoulder joint is the joint between the shoulder bone and the humerus, and is also referred to as the shoulder in a narrow sense. The shoulder joint is the synovial joint made by the contact of the glenoid cavity of the shoulder bone with the humerus head, and is a key part connecting the torso with the arm.

The shoulder is composed of several joints, has the largest motion range (movement range) in the body, and is the farthest away from the hand. Thus, the shoulder functions to reach out the hand to various spaces, thus enabling a person to do various works. Particularly, the shoulder plays an important role in stretching out the hand away from the body or moving the hand behind the body or head. If there is trouble in the shoulder joint, a propulsive force required to take exercise may be decreased or it may be difficult to take accurate exercise.

When the shoulder joint is injured, according to the extent of injury, a plaster cast may be put on the shoulder joint or the arm may be slung by a shoulder brace made of metal and plastics or by a shoulder strap to prevent the arm from sagging or prevent the subluxation of the shoulder joint. However, such a method is problematic in that the arm is kept in close contact with the body, so that, if this method is used for a lengthy period, joint contracture may occur, namely, the shoulder joint may be tightened, and the armpit or the affected area may come into close contact with the cast or the shoulder brace and thereby be poorly ventilated, thus undesirably leading to a slow recovery.

Further, the abduction angle of the shoulder joint has great effect on a recovery from injury. When the abduction angle of the shoulder joint ranges from 40 to 50°, the humeral head is stably pushed into the scapula without tensioning muscle or ligaments around the shoulder joint, thus preventing a dislocation. In the case of using a support for the purpose of maintaining such an angle, the gravity center of the arm is located at the medial ulnar head, thus pressing nerves and blood vessels passing through the medial ulnar head.

As the related art, Patent Document 1 has proposed a shoulder brace, including a brace body which is mounted to a patient's waist and shoulder, a fixing foam which is fixedly attached to a waist part of the brace body, and an arm support which is fixedly attached to the fixing foam, into which the arm corresponding to the injured shoulder is inserted, and presses and supports the inserted arm using the air.

However, this is problematic in that it is not easy to wear the brace, the abduction and adduction of the shoulder joint are difficult, it is impossible to adjust a length depending on the condition of a wearer's body or shoulder, and it is impossible to limit or adjust the motion range of the shoulder joint and the elbow joint, thus making it inconvenient to operate and use.

### SUMMARY OF THE INVENTION

Accordingly, the present invention has been made keeping in mind the above problems occurring in the related art, and an aspect of the present invention is directed to a shoulder brace, which guarantees easy wearing, facilitates both the abduction and the adduction of the shoulder joint, enables both the internal rotation and the external rotation of the elbow joint, is applicable to various body sizes, allows the lengths of a forearm part, an upper-arm part, and a torso part to be adjusted depending on a wearer's body condition, thus being convenient to use, and limits or adjusts the motion ranges of the shoulder joint and the elbow joint depending on the condition of a wearer's shoulder, thus being capable of safely protecting the shoulder and the arm.

In order to accomplish the object, the present invention provides a shoulder brace, including a forearm part worn to surround a forearm; an upper-arm part worn to surround an upper arm; a torso part worn to surround a side of a torso; an elbow-joint part to which the forearm part and the upper-arm part are fastened; and a shoulder-joint part to which the upper-arm part and the torso part are fastened, wherein the upper-arm part may include a rotary annulus to be coupled to an outside of the shoulder-joint part, the rotary annulus having line-shaped protrusions and grooves that are alternately formed along an inner circumference thereof, and the shoulder-joint part may include a joint shaft; a push button provided on a first end thereof and coupled with the joint shaft; a locking ring coupled to a middle portion of the joint shaft and having line-shaped protrusions and grooves that are alternately formed along an outer circumference thereof, so that the locking ring is locked and fastened to the inner circumference of the rotary annulus; and a stopper formed on a second end thereof, and providing space to allow the joint shaft to be movable when the push button is pushed, thus allowing a lock state to be released, whereby, if a lock button is pushed, the locking ring may be unlocked from the rotary annulus while the joint shaft moves, thus allowing the upper-arm part to be rotatably moved.

The present invention is advantageous in that it guarantees easy wearing, facilitates both the abduction and the adduction of the shoulder joint, enables both the internal rotation and the external rotation of the elbow joint, is applicable to various body sizes, allows the lengths of a forearm part, an upper-arm part, and a torso part to be adjusted depending on a wearer's body condition, thus being convenient to use, and limits or adjusts the motion ranges of the shoulder joint and the elbow joint depending on the condition of a wearer's shoulder, thus being capable of safely protecting the shoulder and the arm.

### BRIEF DESCRIPTION OF THE DRAWINGS

The above and other aspects, features and advantages of certain exemplary embodiments of the present invention will be more apparent from the following description taken in conjunction with the accompanying drawings, in which:

FIG. 1 is a perspective view illustrating a shoulder brace, according to the present invention;

FIGS. 2A and 2B are views illustrating a shoulder-joint part when seen from different directions, FIG. 2C is an exploded perspective view of the shoulder-joint part, FIG. 2D is a view illustrating the shoulder-joint part in an unlock state when a push button is pushed, FIG. 2E is a view illustrating the abduction and adduction of the shoulder-joint part when the shoulder-joint part is in the unlock state, FIG. 2F is a view illustrating a configuration related with the motion range of the shoulder-joint part;

FIG. 3A is a perspective view illustrating a forearm part, an upper-arm part, and an elbow-joint part, FIG. 3B is an exploded perspective view illustrating the forearm part, the upper-arm part, and the elbow-joint part, FIG. 3C is a view illustrating the length adjustment of an arm adjuster, FIGS. 3D and 3E are views illustrating restrictions on the motion range of the elbow-joint part, FIG. 3F is a view illustrating an operation of the shoulder-joint part when the shoulder-joint part is in the unlock state; and

FIG. 4A is a perspective view illustrating a torso part, and FIG. 4B is an exploded perspective view illustrating the torso part.

### DETAILED DESCRIPTION OF EXEMPLARY EMBODIMENTS

Exemplary embodiments of the present invention will be described below in detail with reference to the accompanying drawings.

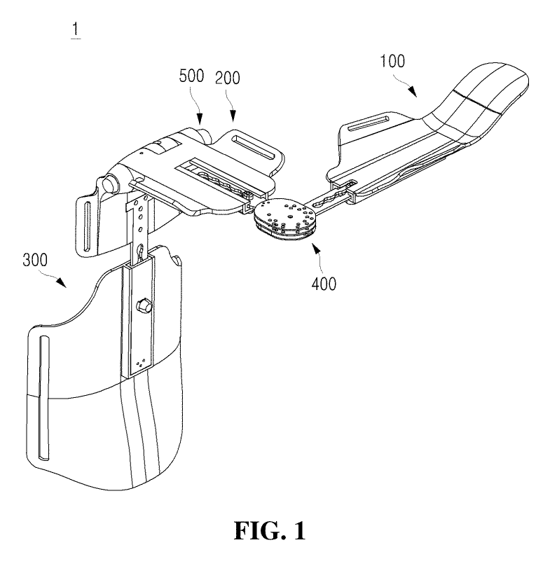

A shoulder brace 1 according to the present invention guarantees easy wearing, is adjustable in length depending on a wearer's body condition, and is intended to limit or adjust the motion range of the shoulder joint and elbow joint depending on a wearer's shoulder condition. Referring to FIG. 1, the shoulder brace 1 includes a forearm part 100 that is worn to surround and protect a medial portion of the forearm, an upper-arm part 200 that is worn to surround and protect a medial portion of the upper arm, a torso part 300 that is worn to surround and protect a side of the torso, an elbow-joint part 400 to which the forearm part and the upper-arm part are fastened, and a shoulder-joint part 500 to which the upper-arm part and the torso part are fastened.

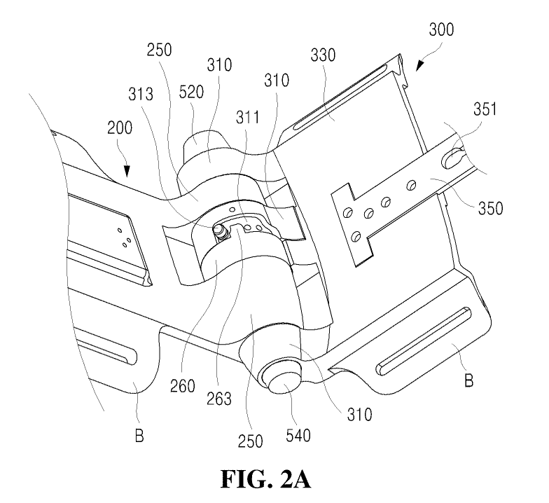

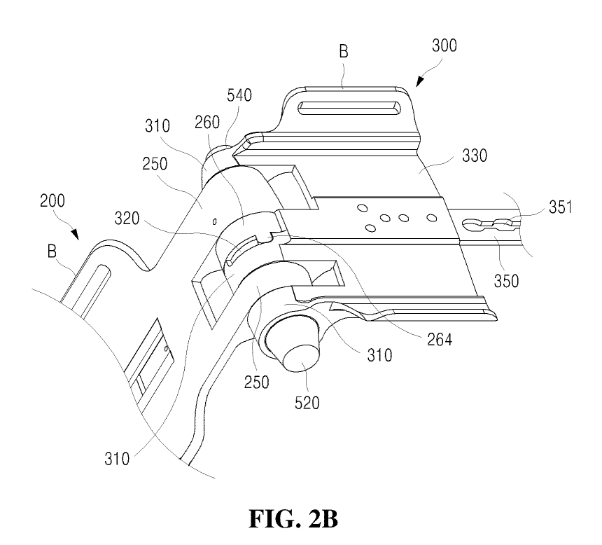

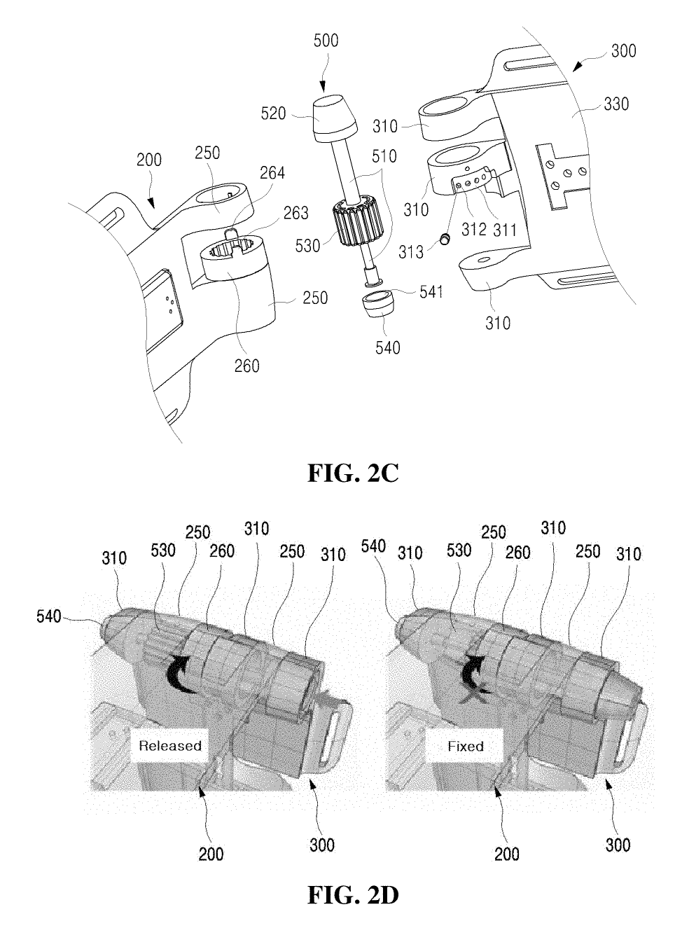

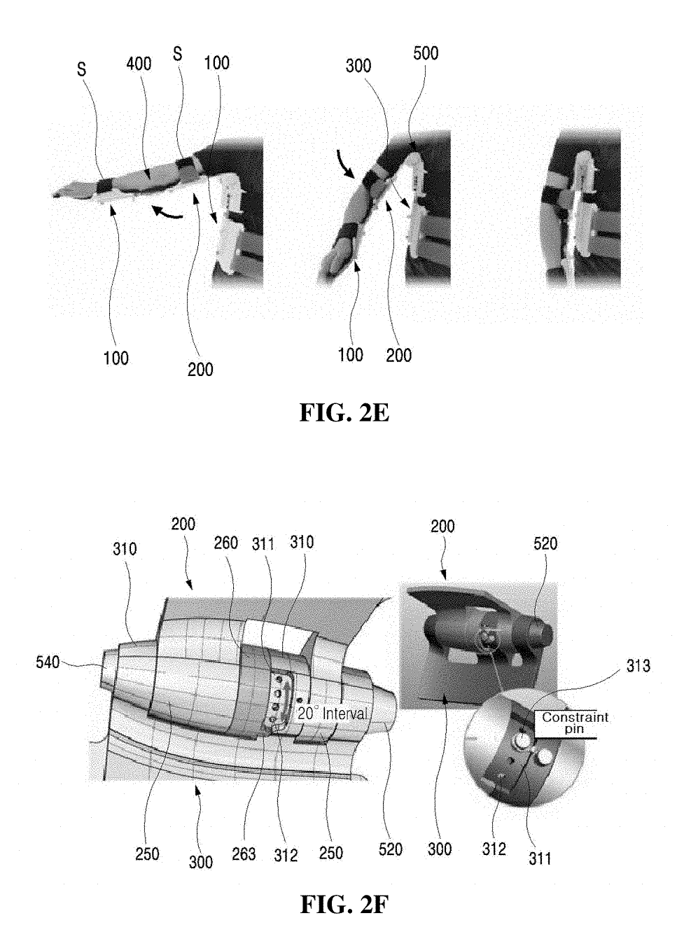

Referring to FIGS. 2A to 2C, the upper-arm part 200 surrounds and protects the medial portion of the upper arm. One rotary annulus or two rotary annuli 250 is or are formed on an end of the upper-arm part 200, so that the rotary annulus or annuli 250 is or are coupled to an outside of the shoulder-joint part 500 to be locked, or is or are unlocked to be rotatable. The rotary annulus 250 is open at an inside thereof to form a hole. The hole has a diameter and a space that are sufficient to accommodate a locking ring 530 when a push button 520 is pushed. The rotary annulus 250 is divided into a forming part and a non-forming part. The forming part is formed on a portion of an inner circumference of the rotary annulus, and has line-shaped protrusions and grooves that are alternately formed along the inner circumference side by side. The non-forming part having no protrusion and groove is formed on a remaining portion of the inner circumference. A rotary ring 260 is connected to a side of the rotary annulus 250 to be rotatable along with the rotary annulus 250. The rotary ring 260 has line-shaped protrusions and grooves that are alternately formed on an inner circumference thereof, similarly to the rotary annulus 250.

Thus, the locking ring 530, fixed by the engagement of the protrusions and the grooves formed throughout the forming part of the rotary annulus 250 and the rotary ring 260, moves to the non-forming part when the push button 520 is pushed, thus releasing a lock state.

Here, the protrusions and the grooves are formed in a line shape in a direction from front to back. In this regard, the direction from front to back means a direction in which a wearer's front is a front side and a wearer's back is a back side when he or she wears the shoulder brace.

Referring to FIG. 2c, the shoulder-joint part 500 has a joint shaft 510 arranged in the direction from front to back, a push button 520 formed on one end of the shoulder-joint part 500 to be coupled with and moved along with the joint shaft 510, and the locking ring 530 coupled to a middle portion of the joint shaft 510. Line-shaped protrusions and grooves are alternately formed on an outer circumference of the locking ring 530 side by side so that the outer circumference of the locking ring is locked and fastened to the inner circumference of the rotary annulus 250. Here, the protrusions and grooves are formed in a line shape in the direction from front to back. A stopper 540 is formed on the other end of the shoulder-joint part 500 to be connected with a fastening ring 310. A stopper groove 541 is formed in the stopper 540, thus providing a space in which the joint shaft 510 coupled with the push button 520 is movable when the push button 520 is pushed, and allowing the lock state to be released.

To be more specific, referring to FIG. 2D, the outer circumference of the locking ring 530 formed on a middle portion of the joint shaft 510 is fixed to the inner circumferences of the rotary annulus 250 and the rotary ring 260 via the engagement of the protrusions and the grooves, thus maintaining the lock state. In such a lock state, the upper-arm part 200 is not rotated. At this time, if a wearer pushes the push button 520 with the hand of the free arm, the locking ring 530 slides along with the joint shaft 510 to be removed from the rotary annulus 250 and moved to an internal space of the rotary annulus, thus releasing the lock state, and an end of the joint shaft 510 moves to the stopper groove 541. In such a release state, as illustrated in FIG. 2E, both the abduction and the adduction of a wearer's shoulder joint are facilitated, thus allowing the upper-arm part 200 to be rotated. Thereafter, if force acting on the push button 520 is removed, the locking ring 530 is locked to the inside of the rotary annulus 250 and then is restored to its original state.

Further, a rotating protrusion 263 of the rotary ring 260 protrudes into an adjusting groove 311. In contrast, the torso part 300 has one fastening ring 310 or two or more fastening rings 310. The fastening ring 310 is coupled to surround an exterior of the shoulder-joint part 500 while making contact with the rotary annulus 250. The fastening ring 310 has the adjusting groove 311 formed on a portion of the outer circumference thereof. A plurality of adjusting holes 312 is formed on the adjusting groove 311 to be spaced apart from each other. A constraint pin 313 is inserted into an adjusting hole 312 selected by a wearer.

For example, referring to FIG. 2F, four adjusting holes 312 are formed to be spaced apart from each other at an angular interval of 20°. Since the movement of the rotating protrusion 263 is constrained to 60° or less depending on a location to which the constraint pin 313 is inserted and thus the motion range of the shoulder joint is limited to 60° or less, it is possible to protect the shoulder without tensioning or damaging muscle or ligaments around the shoulder joint, in addition to allowing the remedial exercise of the shoulder.

In addition, a guide groove 320 having the same shape as the adjusting groove 311 is formed at a position opposite to a position where the adjusting groove 311 of the fastening ring 310 is formed, and a guide protrusion 264 having the same shape as the rotating protrusion 263 is formed to protrude into the guide groove 320 at a position opposite to a position where the rotating protrusion 263 of the rotary ring 260 is formed. Thus, when the rotating protrusion 263 moves along with the rotary ring 260 by the rotation of the upper-arm part 200 or stops moving, the guide protrusion 264 moves along with the rotating protrusion or stops moving, thus guiding an operation or a stop.

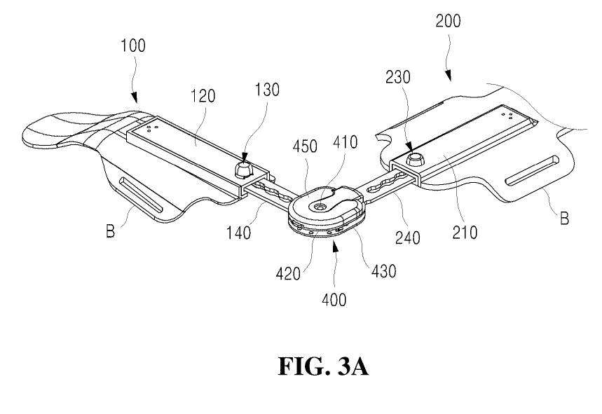

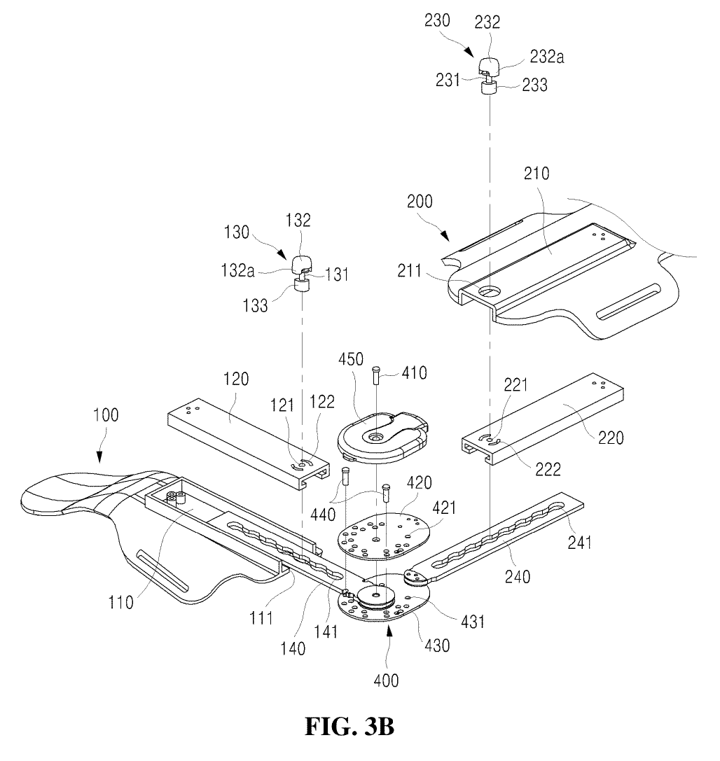

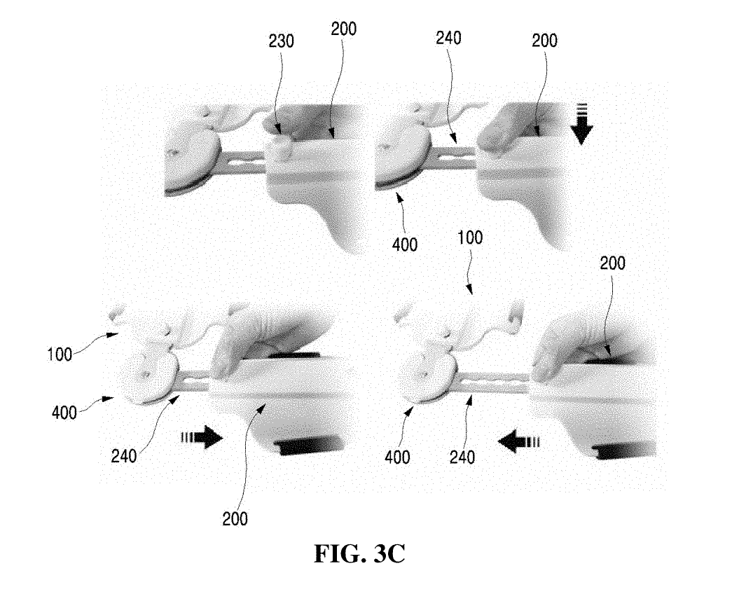

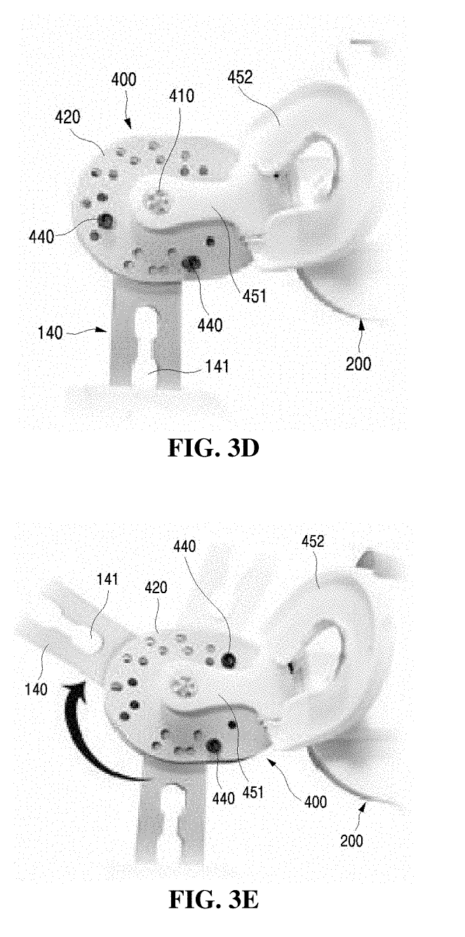

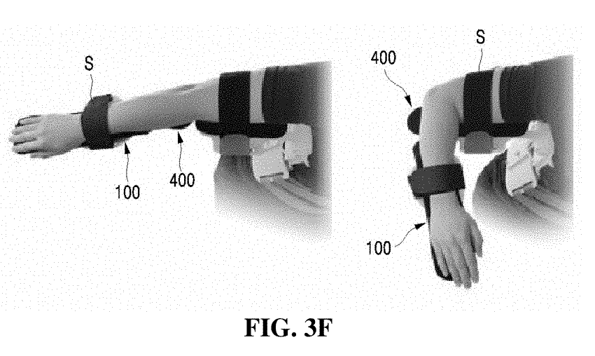

Further, referring to FIGS. 3A and 3B, the forearm part 100 has a slot 110 that is open at a top thereof, and the upper-arm part 200 has a slot 210 that is open at a bottom thereof. The slot 110 of the forearm part 100 has a bottom hole 111 that is formed through a bottom surface thereof. The slot 210 of the upper-arm part 200 has an upper hole 211 that is formed through an upper surface thereof. Rail members 120 and 220 are fastened to the slots 110 and 210, respectively. Button holes 121 and 221 are formed through the rail members 120 and 220, respectively. Push buttons 130 and 230 are fastened to the button holes 121 and 221, respectively. Arc-shaped guide holes 122 and 222 are formed through the rail members 120 and 220 to be located outside the button holes 121 and 221, respectively. Portions of button heads 132 and 232 extend downwards, thus forming lower protrusions 132a and 232a. If the push buttons 130 and 230 are pushed, the lower protrusions 132a and 232a are inserted into the guide holes 122 and 222 while the movement of the push buttons 130 and 230 is guided.

In this regard, the push buttons 130 and 230 include push shafts 131 and 231, button heads 132 and 232 that are formed on upper ends of the push shafts 131 and 231 to be pushed, and fixing plates 133 and 233 that are formed on lower ends of the push shafts 131 and 231, have a diameter larger than that of each push shaft, and are rounded at outer circumferences thereof.

Further, arm adjusters 140 and 240 having a plurality of adjusting holes 141 and 241 that are formed in a longitudinal direction to communicate with each other are entirely or partially inserted into the rail members 120 and 220 of the forearm part 100 and the upper-arm part 200, respectively. As illustrated in FIG. 3C, the fixing plates 133 and 233 of the push buttons 130 and 230 are inserted into adjusting holes 141 and 241 selected by a wearer to maintain the fixed state. In such a state, if the wearer pushes the push buttons 130 and 230, the push buttons 130 and 230 move downwards and the fixing plates 133 and 233 move downwards through the bottom hole 111 of the forearm part 100 or an opening formed in a bottom of the upper-arm part 200, so that the fixed state of the arm adjusters 140 and 240 is released, thus allowing the arm adjusters 140 and 240 to be slidably moved. After the lengths of the arm adjusters 140 and 240 are adjusted depending on the condition of a wearer's body or arm by sliding the am adjusters, force acting on the push buttons 130 and 230 is eliminated. Then, the push buttons 130 and 230 move upwards to return to their original positions, and the fixing plates 133 and 233 are coupled to other desired adjusting holes 141 and 241, thus allowing the brace to be conveniently used with a newly adjusted length. As such, it is possible to adjust the lengths of the forearm part 100 and the upper-arm part 200 depending on a wearer's body condition, thus guaranteeing a convenient use.

The elbow-joint part 400 located at the elbow joint includes upper and lower plates 420 and 430 that are provided at upper and lower positions thereof to be spaced apart from each other and are coupled to each other by a rotating shaft 410 passing through the upper and lower plates. A plurality of fixing holes 421 and 431 is formed through the same locations of the upper and lower plates 420 and 430. After a cover plate 450 is rotated and lifted up, two fixing holes 421 and 431 formed at the same locations of the upper and lower plates 420 and 430 are selected and then a pair of fixing pins 440 is inserted into the selected fixing holes. A top of the upper plate 420 is covered by a cover plate 450.

Referring to FIGS. 3D and 3E, the cover plate 450 includes a stationary part 451 that is fixed to the upper plate 420, and a rotary part 452 that has a horseshoe shape and is rotatably connected to the stationary part by inserting protrusions into holes formed on both ends outside the stationary part 451. One end of the arm adjuster 140 connected to the forearm part 100 is inserted into space between the upper plate 420 and the lower plate 430 of the elbow-joint part 400 to be rotatably coupled to the rotating shaft 410. The range of motion is limited by the pair of fixing pins 440 inserted into the fixing holes 421 and 431.

For example, when a pair of fixing pins 440 is inserted into the fixing holes 421 and 431 as illustrated in FIG. 3E, the forearm part 100 connected with the arm adjuster 140 has the range of motion from −130° to 130°. Referring to FIG. 3F, both the internal rotation and the external rotation of the elbow joint are allowed, thus preventing a unidirectional operation of the forearm part 100 and thereby allowing the brace to be applied to both left and right shoulder joints. By adjusting positions into which the pair of fixing pins 440 is inserted depending on the condition of a wearer's arm, it is possible to limit the range of motion and protect the elbow joint.

In addition, one end of the arm adjuster 240 connected to the upper-arm part 200 is inserted into space between the upper plate 430 and the lower plate 440 and then is fixed by a fixing member.

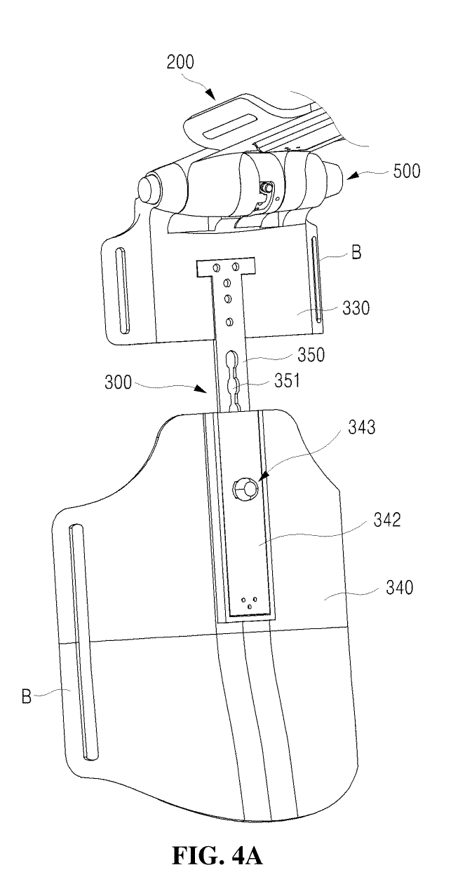

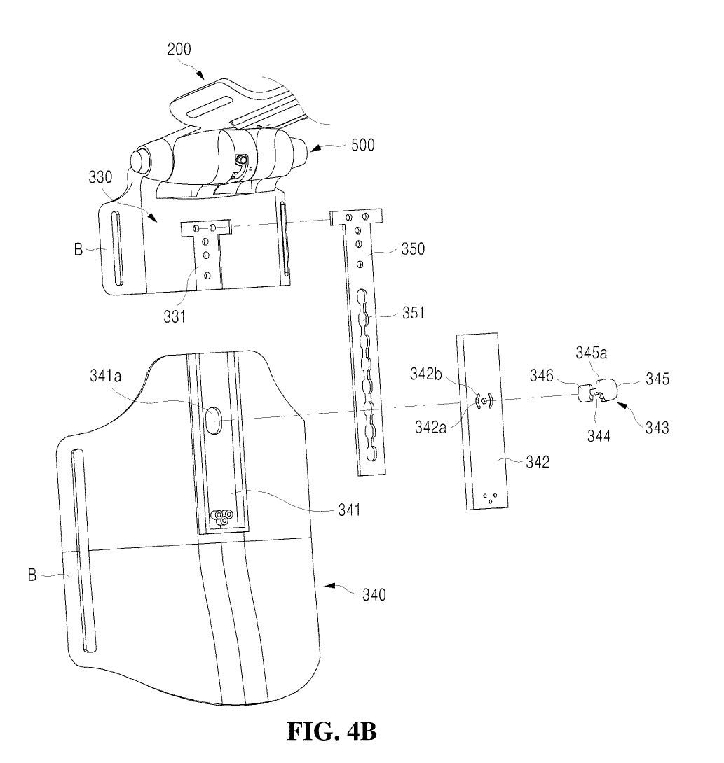

Further, referring to FIGS. 4A and 4B, the torso part 300 includes a first torso plate 330 connected with the shoulder-joint part 500, and a second torso plate 340 arranged to be spaced apart from the first torso plate 330.

The second torso plate 340 includes a slot 341 in which a bottom hole 341a is formed, a rail member 342 having a button hole 342a and fastened to the slot 341, and a push button 343 fastened to the button hole 342a. The push button 343 includes a push shaft 344, a button head 345 formed on an upper end of the push shaft 344, and a fixing plate 346 formed on a lower end of the push shaft 344 and having a diameter larger than that of the push shaft 344. An arc-shaped guide hole 342b is formed through a portion of the rail member 342 outside the button hole 342a. A portion of the button head 345 extends downwards, thus forming a lower protrusion 345a. If the push button 343 is pushed, the movement of the push button 343 is guided while the lower protrusion 345a is inserted into the guide hole 342b.

Moreover, a torso adjuster 350 having a plurality of adjusting holes 351 formed in a longitudinal direction thereof is inserted into the rail member 342 of the second torso plate 340. The fixing plate 346 is fixedly coupled to any adjusting hole 351 selected by a wearer. In such a state, if the wearer pushes the push button 343, the fixing plate 346 moves downwards through the bottom hole 341a and then the fixed state is released. Thus, it is possible to adjust the length by sliding the torso adjuster 350 depending on the condition of a wearer's body or shoulder. A T-shaped fixing groove 331 is formed on an upper portion of the first torso plate 330, and a T-shaped end of the torso adjuster 350 is inserted into the fixing groove 331, thus achieving a firm fixing operation.

In addition, each of the forearm part 100, the upper-arm part 200 and the torso part 300 is provided with a fastening piece B having on front and rear ends thereof fastening holes. By connecting the strap S to the fastening holes, a wearer may wear the brace.

Therefore, the present invention provides a shoulder brace 1, which guarantees easy wearing, facilitates both the abduction and the adduction of the shoulder joint, enables both the internal rotation and the external rotation of the elbow joint, is applicable to various body sizes, allows the lengths of a forearm part, an upper-arm part, and a torso part to be adjusted depending on a wearer's body condition, thus being convenient to use, and limits or adjusts the motion ranges of the shoulder joint and the elbow joint depending on the condition of a wearer's shoulder, thus being capable of safely protecting the shoulder and the arm.

While the invention has been shown and described with reference to exemplary embodiments thereof, it will be understood by those skilled in the art that various changes in form and details may be made therein without departing from the spirit and scope of the invention as defined by the appended claims. Therefore, it is to be understood that the scope of the invention is not limited to the above-described embodiments, and all changes that fall within meets and bounds of the claims, or equivalence of such meets and bounds are intended to be embraced by the claims.

What is claimed is:

1. A shoulder brace, comprising:
a forearm part worn to surround a forearm; an upper-arm part worn to surround an upper arm; a torso part worn to surround a side of a torso; an elbow-joint part to which the forearm part and the upper-arm part are fastened; and a shoulder-joint part to which the upper-arm part and the torso part are fastened,
wherein the upper-arm part comprises a rotary annulus to be coupled to an outside of the shoulder-joint part, the rotary annulus having line-shaped protrusions and grooves that are alternately formed along an inner circumference thereof, and
the shoulder-joint part comprises:
a joint shaft;
a push button provided on a first end thereof and coupled with the joint shaft;
a locking ring coupled to a middle portion of the joint shaft and having line-shaped protrusions and grooves that are alternately formed along an outer circumference thereof, so that the locking ring is locked and fastened to the inner circumference of the rotary annulus; and
a stopper formed on a second end thereof, and providing space to allow the joint shaft to be movable when the push button is pushed, thus allowing a lock state to be released,
whereby, if a lock button is pushed, the locking ring is unlocked from the rotary annulus while the joint shaft moves, thus allowing the upper-arm part to be rotatably moved.

2. The shoulder brace of claim 1, wherein a rotary ring is connected to a side of the rotary annulus to be rotatable along with the rotary annulus, the rotary ring comprising line-shaped protrusions and grooves that are alternately formed along an inner circumference thereof, the rotary annulus comprising a rotating protrusion that is formed to protrude into an adjusting groove,
the torso part comprises a fastening ring to be coupled to an outside of the shoulder-joint part while making contact with the rotary annulus, the fastening ring comprising the adjusting groove formed on an outer circumference thereof, a plurality of adjusting holes being formed in the adjusting groove to be spaced apart from each other, a constraint pin being inserted into a selected adjusting hole, and
the rotating protrusion is constrained by the constraint pin while rotating in the adjusting groove, thus limiting a rotation range of the upper-arm part.

3. The shoulder brace of claim 1, wherein each of the forearm part and the upper-arm part comprises a slot in which a bottom hole is formed, a rail member having a button hole and fastened to the slot, and a push button fastened to the button hole, and the push button comprises a push shaft, a button head formed on an upper end of the push shaft, and a fixing plate formed on a lower end of the push shaft and having a diameter larger than that of the push shaft, and
an arm adjuster having a plurality of adjusting holes formed in a longitudinal direction thereof is inserted into the rail member of each of the forearm part and the upper-arm part, and, if the push button is pushed in a fixed state where the fixing plate is coupled to a selected adjusting hole, the fixing plate moves downwards through the bottom hole to release the fixed state and the arm adjuster is slidably moved, thus allowing a length to be adjusted

4. The shoulder brace of claim 1, wherein the elbow-joint part comprises upper and lower plates that are formed to be spaced apart from each other and are connected by a rotating shaft, with a plurality of fixing holes being formed through each of the upper and lower plates; a pair of fixing pins inserted into selected fixing holes of the upper and lower plates; and a cover plate covering a top of the upper plate, and
the arm adjuster connected to the forearm part is inserted between the upper and lower plates of the elbow-joint part to be rotatably coupled to the rotating shaft, and limits a rotation range by the pair of fixing pins.

5. The shoulder brace of claim 1, wherein the torso part comprises a first torso plate connected with the shoulder-joint part, and a second torso plate arranged to be spaced apart from the first torso plate,
the second torso plate comprises a slot in which a bottom hole is formed, a rail member having a button hole and fastened to the slot, and a push button fastened to the button hole, and the push button comprises a push shaft, a button head formed on an upper end of the push shaft, and a fixing plate formed on a lower end of the push shaft and having a diameter larger than that of the push shaft, and
a torso adjuster having a plurality of adjusting holes formed in a longitudinal direction thereof is inserted into the rail member of the second torso plate, and, if the push button is pushed in a fixed state where the fixing plate is coupled to a selected adjusting hole, the fixing plate moves downwards through the bottom hole to release the fixed state and the torso adjuster is slidably moved, thus allowing a length to be adjusted.

6. The shoulder brace of claim 1, wherein the first torso plate comprises a T-shaped fixing groove formed on an upper portion thereof, and a T-shaped end of the torso adjuster is inserted into the fixing groove to be fixedly fastened thereto.

7. The shoulder brace of claim 3 wherein the rail member comprises an arc-shaped guide hole formed outside the button hole,
a portion of the button head extends downwards to form a lower protrusion, so that, if the push button is pushed, the lower protrusion is inserted into the guide hole, thus guiding a movement of the push button.

8. The shoulder brace of claim 1, wherein each of the forearm part, the upper-arm part and the torso part comprises a fastening piece having fastening holes formed on front and rear ends thereof, so that the brace is worn on a body by connecting a strap to the fastening holes.
